(19) United States
(12) Patent Application Publication
(10) Pub. No.: US 2025/0215007 A1
(43) Pub. Date: Jul. 3, 2025

(54) PREPARATION METHOD FOR HEAT-RESISTANT ENERGETIC MATERIAL PTO

(71) Applicant: Nanjing University of Science and Technology, Nanjing (CN)

(72) Inventors: Yuangang XU, Nanjing (CN); Ming LU, Nanjing (CN); Rongzheng ZHANG, Nanjing (CN)

(73) Assignee: Nanjing University of Science and Technology, Nanjing (CN)

(21) Appl. No.: 18/948,574

(22) Filed: Nov. 15, 2024

(30) Foreign Application Priority Data
Dec. 29, 2023 (CN) 202311854603.3

(51) Int. Cl. C07D 487/04

(52) U.S. Cl. CPC C07D 487/04

(57) ABSTRACT

A preparation method for a heat-resistant energetic material PTO is provided, where PTO is synthesized by a one-step reaction with commercial 1H-pyrazolo[3,4-d]pyrimidin-4-amine as raw material under the reaction of fuming HNO₃ and concentrated H₂SO₄, and the yield is up to 75%. The preparation method is 3 steps shorter than the existing technical route, the reaction time is greatly reduced, the reaction conditions are relatively mild, the process is simpler, and it is easy to scale up in engineering.

### CROSS REFERENCE TO THE RELATED APPLICATIONS

This application is based upon and claims priority to Chinese Patent Application No. 202311854603.3, filed on Dec. 29, 2023, the entire contents of which are incorporated herein by reference.

### TECHNICAL FIELD

The invention relates to a preparation method for a heat-resistant energetic material PTO, which belongs to the technical field of energetic materials.

### BACKGROUND

PTO (4-amino-5-nitro-7H-pyrazolo[3,4-d][1,2,3]triazine 2-oxide) is a heat-resistant energetic material synthesized by the China Academy of Engineering Physics in 2022 (Chem. Eng. J., 2022, 429, 132172). The density of PTO is 1.82 g·cm⁻³, the enthalpy of formation is 343.4 kJ·mol⁻¹, the calculated detonation velocity is 8525 m·s⁻¹, and the calculated detonation pressure is 29.4 GPa, the energetic performance of PTO is close to that of LLM-105, which is significantly higher than that of traditional heat-resistant explosives TATB, PYX, and HNS. PTO has lower mechanical sensitivity than PYX and HNS, and higher thermal decomposition temperature (up to 365° C.), it is an ultra-high temperature heat-resistant energetic material with excellent comprehensive performance and has broad application prospects.

At present, PTO is synthesized from 3-amino-4-cyanopyrazole by four steps of [3+2]cycloaddition with sodium azide, nitration, ammonification, and acidification, the first step requires reflux for 12 h; the second step requires heating at 50° C. for 4 h; the third step requires heating at 60° C. for 8 h; although the yield of each step was high (>85%), the total yield is only 71%. In addition, the product of the second step is a high-energy primary explosive, and its sensitivity is very high (Nat. Comun. 2019, 10, 1339), therefore, the existing reaction route has long steps and high risks, and it is urgent to develop a new simple synthetic method of PTO.

### SUMMARY

The invention overcomes the shortcomings of the existing technology and provides a preparation method for heat-resistant energetic material PTO, and uses commercial raw materials to prepare PTO efficiently by one-step reaction.

A technical solution for realizing the purpose of the invention:

A preparation method for a heat-resistant energetic material PTO includes:

(1) at −5-5° C., adding 1H-pyrazolo[3,4-d]pyrimidin-4-amine to 98 wt % concentrated sulfuric acid (H₂SO₄) in small portions, keeping stirring for a period, and then adding fuming nitric acid (HNO₃) dropwise, slowly heating a reaction solution at a heating rate of 1° C.·min⁻¹ to a predetermined temperature and keeping stirring at this temperature for a predetermined time;

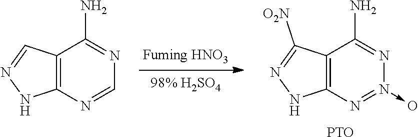

(2) after the reaction, pouring the reaction solution into ice water, filtering to collect a precipitation, washing with cold water, and drying in the air to obtain PTO.

Preferably, in Step (1), a concentration of fuming HNO₃>95 wt %.

Preferably, in Step (1), a ratio of 1H-pyrazolo[3,4-d]pyrimidin-4-amine to 98 wt % concentrated H₂SO₄ is 1 g: 0.5-15 mL, preferably 1 g: 1-7 mL, more preferably 1 g: 2-5 mL; a ratio of 1H-pyrazolo[3,4-d]pyrimidin-4-amine to fuming HNO₃ is 1 g: 0.5-15 mL, preferably 1 g: 4-15 mL, and more preferably 1 g: 4-8 mL.

Preferably, in Step (1), adding 1H-pyrazolo[3,4-d]pyrimidin-4-amine to 98 wt % concentrated H₂SO₄ in small portions at −5-5° C., and stirring for 0.5 h.

Preferably, in Step (1), adding fuming HNO₃ dropwise, and meanwhile controlling a temperature of the reaction solution not to exceed 10° C.

Preferably, in Step (1), slowly heating the reaction solution to 40-80° C. and stirring at this temperature for 2-15 h, a reaction temperature is preferably 60-70° C. and a reaction time is preferably 5-15 h.

Preferably, in Step (2), pouring the reaction solution into ice water with 3-4 times the volume of the reaction solution after the reaction, and filtering and collecting the precipitation within 15 min.

Compared with the existing technology, the beneficial effect of the invention is as follows:

(1) The invention uses a one-pot method to prepare the PTO, the synthetic route only needs one step, which is 3 steps shorter than the existing technical synthetic route, and the reaction time is greatly shortened. The reaction conditions are relatively mild, the process is simpler, and it is easy to scale up.

(2) The highest yield of the new PTO preparation method of the invention is close to 75%, which is significantly higher than the total yield of the existing technology.

### BRIEF DESCRIPTION OF THE DRAWINGS

FIG. 1 is a ¹H NMR spectrum of the PTO in the invention.

FIG. 2 is a ¹³C NMR spectrum of the PTO in the invention.

FIG. 3 is a DSC diagram of the PTO in the invention.

FIG. 4 is a mass spectrum of the PTO in the invention.

FIG. 5 is an infrared spectrum of the PTO in the invention.

FIG. 6 is a single crystal structural diagram of the PTO·DMF in this invention.

FIG. 7 is a cell stacking diagram of the PTO·DMF in the invention.

### DETAILED DESCRIPTION OF THE EMBODIMENTS

The following embodiments enable the professional and technical personnel to understand the invention more comprehensively but do not restrict the invention in any way.

The synthetic route of the heat-resistant energetic material PTO described in the invention is as follows:

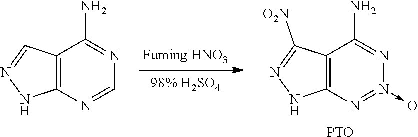

### Embodiment 1

At −5° C., 1H-pyrazolo[3,4-d]pyrimidin-4-amine (1 g, 7.4 mmol, CAS: 2380-63-4) was added to 2 mL H₂SO₄ (98 wt %) in small portions, after stirring for 0.5 h, 5 mL fuming HNO₃ was added dropwise while maintaining the temperature below 5° C. After the addition, the reaction was slowly heated to 60° C. and reacted at this temperature for 6 h. After the reaction was completed, the reaction solution was poured into ice water with 3-4 times the volume of the reaction solution, the precipitation was filtered and collected within 15 min, washed with cold water, and dried in air to obtain 1.09 g of yellow PTO solid, with a yield of 74.7%.

### Embodiment 2

Other conditions are the same as those in Embodiment 1, and the experiments at different temperatures are tested, the experimental results are shown in Table 1.

TABLE 1
Experiments at different temperatures

| No. | Temperature/° C. | Yield of PTO/% |
|---|---|---|
| 1 | 30 | 1.7 |
| 2 | 40 | 15.3 |
| 3 | 50 | 41.7 |
| 4 | 60 (Embodiment 1 ) | 74.7 |
| 5 | 70 | 50.6 |
| 6 | 80 | 29.3 |
| 7 | 90 | 0 |

### Embodiment 3

Other conditions are the same as those in Embodiment 1, and the experiments with different reaction times are tested. The experimental results are shown in Table 2.

TABLE 2
Experiments with different reaction times

| No. | Reaction time/h | Yield of PTO/% |
|---|---|---|
| 1 | 2 | 15.3 |
| 2 | 3 | 32.4 |
| 3 | 4 | 49.1 |
| 4 | 5 | 61.5 |
| 5 | 6 (Embodiment 1) | 74.7 |
| 6 | 7 | 74.5 |
| 7 | 8 | 75.1 |
| 8 | 10 | 74.3 |
| 9 | 15 | 73.9 |

### Embodiment 4

Other conditions are the same as those in Embodiment 1, and the experiments with different dosages of fuming HNO₃ are tested. The experimental results are shown in Table 3.

TABLE 3
Experiments with different amounts of fuming HNO₃

| No. | Dosage of fuming HNO₃/mL | Yield of PTO/% |
|---|---|---|
| 1 | 0.5 | 8.5 |
| 2 | 1 | 19.6 |
| 3 | 2 | 34.1 |
| 4 | 3 | 49.8 |
| 5 | 4 | 67.6 |
| 6 | 5 (Embodiment 1) | 74.7 |
| 7 | 6 | 66.3 |
| 8 | 8 | 60.4 |
| 9 | 10 | 58.9 |
| 10 | 12 | 55.0 |
| 11 | 15 | 53.3 |

### Embodiment 5

The other conditions are the same as Embodiment 1, and the experiments with different dosages of concentrated H₂SO₄ (98 wt %) are tested. The experimental results are shown in Table 4.

TABLE 4
Experiment with different dosages of concentrated H₂SO₄ (98 wt %)

| | Dosage of concentrated | |
|---|---|---|
| No. | H₂SO₄/mL | Yield of PTO/% |
| 1 | One drop | 25 |
| 2 | 1 | 55.4 |
| 3 | 2 (Embodiment 1) | 74.7 |
| 4 | 3 | 69.9 |
| 5 | 4 | 68.5 |
| 6 | 5 | 65.8 |
| 7 | 7 | 52.6 |
| 8 | 9 | 44.4 |
| 9 | 11 | 30.3 |
| 10 | 13 | 27.6 |
| 11 | 15 | 16.5 |

The above-mentioned examples are only the better embodiments of the invention, not any formal restriction on the invention. Any technical personnel familiar with the profession, within the scope of the technical scheme of the invention, according to the technical essence of the invention, any simple modification, equivalent replacement, and improvement of the above embodiment are still within the protection scope of the technical scheme of the invention.

### Comparison Case 1:

At −5° C., 1H-pyrazolo[3,4-d]pyrimidin-4-amine (1 g, 7.4 mmol) was added in small portions to 2 mL H₂SO₄ (98 wt %), stirred for 0.5 h, and then 5 mL fuming HNO₃ was added dropwise while maintaining the temperature below 5° C. After the addition, the reaction was slowly heated to 20° C. and reacted at this temperature for 6 h. After the reaction, the reaction solution was poured into ice water with 3-4 times the volume of the reaction solution, no product was precipitated, and the reaction failed.

### Comparison Case 2:

At −5° C., 1H-pyrazolo[3,4-d]pyrimidin-4-amine (1 g, 7.4 mmol) was added in small portions to 2 mL H₂SO₄ (98 wt %), stirred for 0.5 h, and then 5 mL fuming HNO₃ was added dropwise while maintaining the temperature below 5° C. After the addition, the reaction was slowly heated to 90° C. and reacted at this temperature for 6 h. After the reaction, the reaction solution was poured into ice water with 3-4 times the volume of the reaction solution, no product was precipitated, and the reaction failed.

### Comparison Case 3:

At −5° C., 1H-pyrazolo[3,4-d]pyrimidin-4-amine (1 g, 7.4 mmol) was added in small portions to 2 mL H₂SO₄ (98 wt %), stirred for 0.5 h, and then 5 mL fuming HNO₃ was added dropwise while maintaining the temperature below 5° C. After the addition, the reaction was slowly heated to 60° C. and reacted at this temperature for 6 h. After the reaction, the reaction solution was poured into ice water with 3-4 times the volume of the reaction solution, and the precipitate was filtered and collected within 15 min. The yield was less than 10%. It was found by TLC that the product had unreacted 1H-pyrazolo[3,4-d]pyrimidin-4-amine and an unknown impurity in addition to PTO.

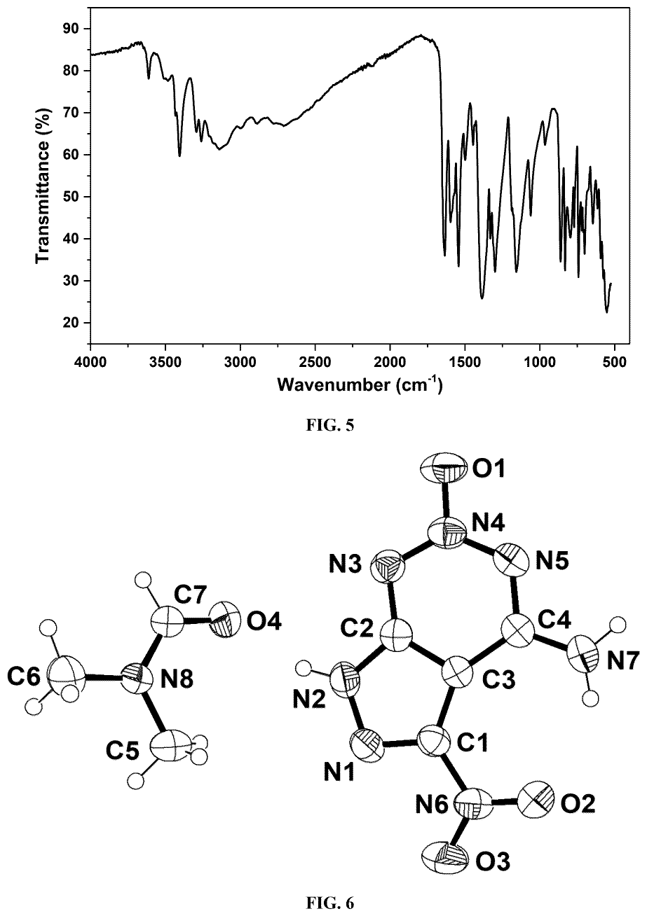

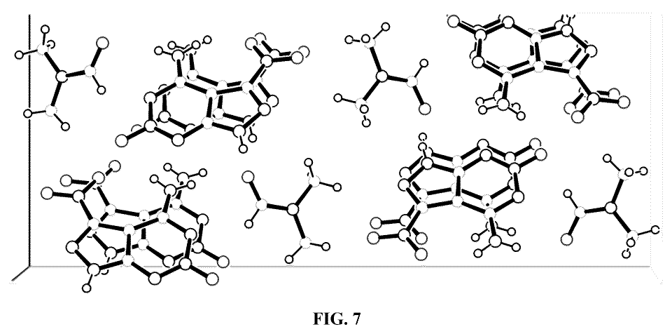

The PTO obtained from Embodiments 1-5 was dissolved in DMF and slowly evaporate at room temperature to obtain the yellow block single crystal. Among them, the single-crystal X-ray diffraction test was carried out on the single crystal of the sample mentioned in Embodiment 1. The crystal structure (PTO·DMF) is shown in FIG. 6 and FIG. 7, and the cell parameters are as follows:
Crystal system: monoclinic crystal system;
space group: P2₁/c (number 14);
cell parameters: a=4.3168(2) Å, b=25.5098(12) Å, c=10.5562(6) Å, α=90°, β=95.647(2)°, γ=90°;
crystal cell volume: 1156.81(10) Å³;
Z=4;
density: 1.552 g·cm⁻³ (296 K).

The PTO obtained from Embodiments 1 to 5 was characterized, and the spectral data were consistent, the analysis results of the samples described in Embodiment 1 are given below, which are consistent with the characterization data reported in the literature.

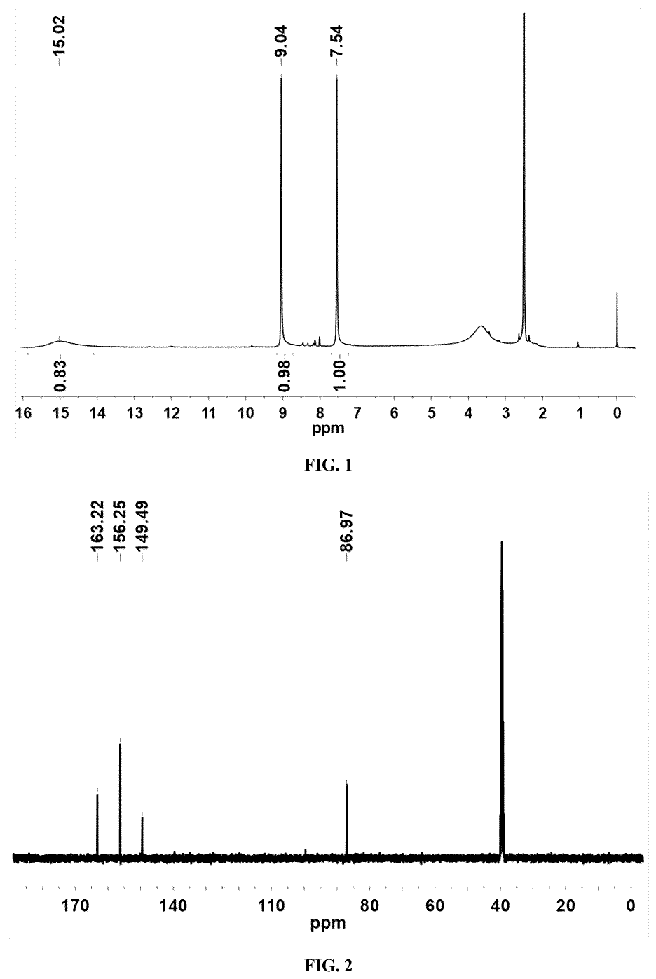

¹H NMR (500 MHz, DMSO-d₆): δ 15.02, 9.04, 7.54 ppm, as shown in the ¹H NMR spectrum of FIG. 1.

¹³C NMR (125.72 MHz, DMSO-d₆): δ 163.22, 156.25, 149.49, 86.97 ppm, as shown in the ¹³C NMR spectrum of FIG. 2.

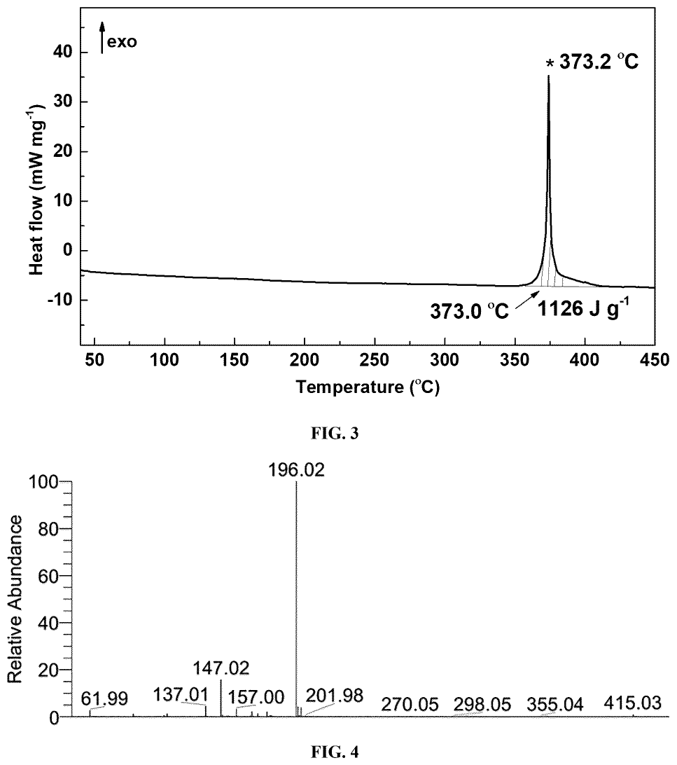

The decomposition temperature is 373.0° C. and the exothermic peak temperature is 373.2° C., as shown in the DSC diagram of FIG. 3.

m/z: 196.02 (M-H), as shown in the mass spectrum of FIG. 3.

IR (ATR): \tilde{ν} 3611, 3405, 3293, 3260, 3140, 1634, 1596, 1542, 1498, 1445, 1386, 1330, 1299, 1157, 1060, 965, 861, 831, 796, 770, 741, 721, 701, 646, 615, 593, 552 cm⁻¹, as shown in infrared spectrum of FIG. 5.

Elemental analysis for C₄H₃N₇O₃ (197.114): Calculated value (measured value) C, 24.37 (24.31), H, 1.53 (1.55), N, 49.74 (49.67).

What is claimed is:

1. A preparation method for a heat-resistant energetic material 4-amino-5-nitro-7H-pyrazolo[3,4-d][1,2,3]triazine 2-oxide (PTO), comprising steps of:
(1) at −5-5° C., adding 1H-pyrazolo[3,4-d]pyrimidin-4-amine to 98 wt % concentrated H₂SO₄ in small portions, keeping stirring for a period, then adding fuming HNO₃ dropwise, and slowly heating a reaction solution at a heating rate of 1° C.·min⁻¹ to a predetermined temperature and keeping stirring at the predetermined temperature for a predetermined time, 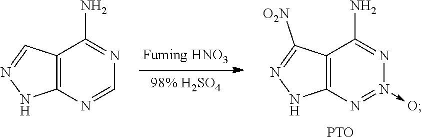
(2) after a reaction in the step (1), pouring the reaction solution into ice water to obtain a mixture, filtering the mixture to collect a precipitation, washing the precipitation with cold water to obtain a washed precipitation, and drying the washed precipitation in air to obtain the heat-resistant energetic material PTO.

2. The preparation method according to claim 1, wherein in the step (1), a concentration of the fuming HNO₃ is >95 wt %.

3. The preparation method according to claim 1, wherein in the step (1), a ratio of the 1H-pyrazolo[3,4-d]pyrimidin-4-amine to the 98 wt % concentrated H₂SO₄ is 1 g:(0.5-15) mL; a ratio of the 1H-pyrazolo[3,4-d]pyrimidin-4-amine to the fuming HNO₃ is 1 g:(0.5-15) mL.

4. The preparation method according to claim 1, wherein the step (1) comprises adding the 1H-pyrazolo[3,4-d]pyrimidin-4-amine to the 98 wt % concentrated H₂SO₄ in the small portions at −5-5° C., and keeping stirring for the period of 0.5 h.

5. The preparation method according to claim 1, wherein the step (1) comprises adding the fuming HNO₃ dropwise, and meanwhile controlling a temperature of the reaction solution not to exceed 10° C.

6. The preparation method according to claim 1, wherein the step (1) comprises slowly heating the reaction solution at the heating rate of 1° C.·min⁻¹ to the predetermined temperature of 40-80° C. and keeping stirring at the predetermined temperature of 40-80° C. for the predetermined time of 2-15 h.

7. The preparation method according to claim 1, wherein the step (2) comprises pouring the reaction solution into the ice water with 3-4 times a volume of the reaction solution after the reaction in the step (1), and filtering and collecting the precipitation within 15 min.

8. The preparation method according to claim 1, wherein in the step (1), a ratio of the 1H-pyrazolo[3,4-d]pyrimidin-4-amine to the 98 wt % concentrated H₂SO₄ is 1 g:(1-7) mL; a ratio of the 1H-pyrazolo[3,4-d]pyrimidin-4-amine to the fuming HNO₃ is 1 g:(4-15) mL.

9. The preparation method according to claim 1, wherein in the step (1), a ratio of the 1H-pyrazolo[3,4-d]pyrimidin-4-amine to the 98 wt % concentrated H₂SO₄ is 1 g:(2-5) mL; a ratio of the 1H-pyrazolo[3,4-d]pyrimidin-4-amine to the fuming HNO₃ is 1 g:(4-8) mL.

10. The preparation method according to claim 1, wherein the step (1) comprises slowly heating the reaction solution at the heating rate of 1° C.·min⁻¹ to the predetermined temperature of 60-70° C. and keeping stirring at the predetermined temperature of 60-70° C. for the predetermined time of 5-15 h.
